(19) United States
(12) Patent Application Publication
(10) Pub. No.: US 2024/0207112 A1
(43) Pub. Date: Jun. 27, 2024

(54) Leak-Proof Seamless Underwear having a Fabric-Made Leak-Protection Pad

(71) Applicants: Delta Galil Industries Ltd., Caesarea (IL); Great Pacific Holdings Limited, Central (HK)

(72) Inventor: Ralph Roland Jansen, Molfsee (DE)

(21) Appl. No.: 18/288,193

(22) PCT Filed: Apr. 27, 2022
(86) PCT No.: PCT/IL2022/050427

§ 371 Date: Oct. 25, 2023

Related U.S. Application Data
(60) Provisional application No. 63/180,663, filed on Apr. 28, 2021.

(51) Int. Cl. A61F 13/66; A61F 13/72; A61F 13/84; A61L 15/18; A61L 15/46

(52) U.S. Cl. CPC A61F 13/665; A61F 13/72; A61L 15/18; A61L 15/46; A61F 2013/8414

(57) ABSTRACT

Leak-proof seamless underwear having a fabric-made leak-protection pad. An underwear for a female wearer includes: a rear-side fabric-region that is configured to cover at least some of buttocks of the female wearer; a front-side fabric-region that is generally opposite said rear-side fabric-region and that is configured to cover at least some of a pelvis of the female wearer; and a gusset (or crotch-covering) fabric-region that is configured to cover genitalia and/or crotch of the female wearer. A body-facing or inner side of the gusset fabric-region includes a fabric-based Passepartout frame element which holds in place therein a fabric-based leak-protection pad that absorbs fluids and prevents fluid leaks.

### CROSS-REFERENCE TO RELATED APPLICATIONS

This patent application claims priority and benefit from U.S. 63/180,663, filed on Apr. 28, 2021, which is hereby incorporated by reference in its entirety.

### FIELD

Some embodiments are related to the field of clothing.

### BACKGROUND

Clothing articles and clothes are typically formed of textile material and are worn on the body. Clothes are worn for various purposes, for example, to keep the human body warm in a cold weather, to protect the human body from rough surfaces or insects or plants, to provide a hygienic barrier between the human body and the environment, to protect the human body from ultraviolet radiation, to cover genitals, for social reasons or as fashion, or the like.

### SUMMARY

Some embodiments provide garments or undergarments, particularly underwear or panties or lingerie items for females, having a leak protection pad that is made or fabric.

In some embodiments, an underwear for a wearer comprises a fabric-made Passepartout frame element, that is attached or stitched or bonded to a crotch or gusset region of the underwear. The fabric-made Passepartout frame element holds in place a fabric-made leak-protection pad, which comprises at least a silver ionized fabric structure. The silver ionized fabric structure is knitted from (i) one or more polyester yarns, and (ii) one or more yarns that are augmented treated with ionized silver molecules. The silver ionized fabric structure does not directly touch the skin of the wearer. The silver ionized fabric structure is sandwiched between (I) a top-side fabric layer that is in direct touch with the skin of the wearer, and (II) a bottom-side leakage-protection membrane or foil, that is non-removably laminated to said silver ionized fabric structure.

In some embodiments, optionally, the fabric-made pad or one or more parts thereof may be removable from the underwear, and may be washed or cleaned and then re-used by re-insertion into the underwear. In other embodiments, the fabric-made pad is non-removable from the underwear.

In some embodiments, the pad is a seamless fabric element that lacks any seams, or that lacks seams at least on a first side that is inserted to be facing upwardly towards the wearer's body, or may lack seams in (or on, or at) any portion of the fabric-made pad that direct touches the wearer's body.

Some embodiments may provide other and/or additional advantages and/or benefits.

### BRIEF DESCRIPTION OF THE DRAWINGS

FIG. 1 is a schematic illustration of a multiple-layer fabric-made (or fabric-based) pad, in accordance with some demonstrative embodiments.

FIGS. 2A-2C are schematic illustrations of several views of an underwear for a female wearer, in accordance with some demonstrative embodiments.

FIG. 3 is a schematic illustration of a fabric-based leak-proof pad, demonstrating the Passepartout element or member, in accordance with some demonstrative embodiments.

### DETAILED DESCRIPTION OF SOME DEMONSTRATIVE EMBODIMENTS

Some embodiments may include garments or undergarments for females, and particularly underwear for females, having a leak protection pad that is made or fabric.

The Applicants have realized that conventional underwear for females lack a leak protection element, and require many females to add or utilize a disposable one-time leak protection pad that is formed of non-fabric materials. Such disposable pads, realized the Applicants, are not environmentally friendly, may cause allergic reaction to some wearers, may cause irritation to some wearers, may not be entirely leak-proof for some wearers, and/or may suffer from other disadvantages or limitations.

In accordance with some embodiments, the fabric-made pad may include multiple layers. For example, a First Layer of the fabric-made pad is formed of Polyester knitted fabric, optionally with a rip knitting effect, which transports liquid away from the body (e.g., within two seconds, as a high wicking element). The rip effect may create uneven or non-planar surfaces or regions in this layer, similar to spatial mountain and valleys; which in turn may hasten the drying of the first layer, or may contribute to (or provide) a fast-dry effect.

A Second Layer if formed of fabric, knitted from a combination of: a first number (P) of Polyester yarn(s), and a second number (S) of silver yarn(s). The Silver yarn(s) may be, for example, a yarn that is augmented with silver ions. In some embodiments, P is 1 and S is 1. In other embodiments, P is 1 and S is 2. In other embodiments, P is 2 and S is 1. In some embodiments, P is a number in the range of 1 to 10, including these values. In some embodiments, S is a number in the range of 1 to 10, including these values. The polyester yarn(s) and the silver ions yarn(s) are knitted together and engineered to create sufficient space for holding a large amount of liquid within the fabric.

In some embodiments, the utilization of silver ions augmented yarn(s) in combination with polyester yarns, may create an anti-bacterial effect, which is not temporary and may last for months or even years, and does not “wash off” in by laundry or washing.

In some embodiments, a breathable membrane is further included in the fabric-made pad; for example, laminated or glued or otherwise attached to the bottom-side of the fabric that includes the polyester yarn(s) and the silver ion augmented yarn(s). The membrane and that fabric, once laminated or glued together, are characterized as being a single laminated element, and cannot be separated from each other anymore (e.g., without destroying the fabric or the functionality of its components), and are provided as a single fabric-made element or a single fabric-made pad.

The fabric-made pad of some embodiments may have various differences from (and advantages over) conventional pads that attempted to achieve similar results.

First, the fabric-made pad of some embodiments is a single unified element, that includes therein both the fabric (made of a knitted combination of silver ion augmented yarn(s) with polyester yarn(s), as described) and the membrane laminated to it in a non-separable way. The membrane layer, in some embodiments, cannot and does not move or shift or slide in its position, relative to the fabric-made pad element, or relative to the body of the human wearer; and thus it provides secure and non-moving protection and the relevant places.

Second, in some embodiments, the fabric-made pad lacks, and excludes, and does not include, any anti-bacterial coating or finishing or washing, which some conventional products has attempted to use. The Applicants have realized that such anti-bacterial washing or finishing or coating is only temporal or temporary, and typically vanishes or dissolves or disappears or significantly weakens within days or few weeks, and/or after several washing or laundry cycles. Rather, the anti-bacterial functionality of the fabric-made pad is achieved via the unique knitting combination of the polyester yarn(s) with the silver ionized yarn(s) or the silver ion augmented yarn(s).

Third, the fabric-made pad in some embodiments includes Permanent non-washing non-disappearing anti-bacterial effect (and also anti-odor/anti-stink effect) and anti-bacterial agent and anti-bacterial yarn(s), in the second layer fabric due to the knitted integration of silver ionized yarn(s) or of yarn(s) that were treated or augmented with silver ions or other silver-containing molecules. Furthermore, the anti-bacterial and anti-odor functionality of the fabric-made pad occurs at the second layer or the middle layer, where the liquid or the wetness are held for the longest period of time; and not in a layer that directly touches the skin of the wearer.

Fourth, some conventional protection pads attempt to provide some leakage protection by utilizing three different and separate and independent layers: (i) a high-wicking body-touching fabric layer, which is the upper layer, with an anti-bacterial coating or washing or finishing that washes-out or washes-away or disappears or weakens significantly after several laundry washes or cycles; (ii) a middle layer made of fleece, for absorbing the liquid and holding it there; (iii) a third layer, which is the bottom layer or the lower layer, which is independent from the other layers and is separate from them, and which is a membrane or a foil that attempts to prevent liquid leakage. In such conventional products, the three layers are stitched together into the underwear, or are bonded at the time when the leg-opening of the underwear goes into a finishing process by a folded-fabric-band with a heat-glue process commonly referred to as “bonding”.

In contrast, some embodiments provide an independent fabric-made leakage-proof pad, that is attached (or, that only needs to be attached) to the underwear at its four corners, or through a conventional sewing operation around the leg openings.

In some embodiments, in addition to the ionized silver that is integrated and knitted into the polyester structure of the fabric-made pad, optionally, anti-bacterial and/or anti-viral and/or anti-fungal treatment or coating or agent or substance may be added to the middle layer or central layer or inner layer of the fabric-made pad, such that the coating or agent or substance or treatment does not directly touch the skin of the wearer; in order to optionally provide some additional anti-bacterial and/or anti-viral and/or anti-fungal functionalities to the pad.

In some embodiments, the fabric layers are sandwiched into or enclosed within a Passepartout element or member, thereby creating an independent self-sustaining fabric-made pad, that provides various options of being attached to the underwear, via sewing or bonding.

### Construction of the Passepartout Pad:

The Two layers of fabric are laminated/bonded together, through two Passepartout frames or borders or enclosures; such that the two layers are independently sandwiched into a Frame; one Passepartout member is located at the Bottom side, and another Passepartout member is located at the Top side; while holding and keeping the middle layers of fabric (with the silver ionized yarns, and with the membrane laminated beneath) open for maximum absorbance of liquid or leakage. The frame provides an entire encapsulation of the leak-proof element, and provides an improved and more secure leakage-proofing mechanism. The Passepartout element operates to fully seal and trap the liquid with the adhesive; once laminated together, the fabric-made pad becomes an independent component, that will contain the liquid, not allowing anything to leak out.

The fabric-made Pad with its Passepartout has a frame around the outside region or border or margin of the fabric layers, which further allows stitching, ultra-sonic treatment, and/or bonding of the fabric-made Pad into the undergarment; such as, utilizing an elastic that may be folded around the leg opening including the outer-extension of the Passepartout. This unique structure may allow integration of the fabric-made pad into a seamless or seam-free underwear (or other undergarment or garment, such as pants or leggings).

Some embodiments may optionally include or utilize a gusset seam, located (for example) across the back-side of a seamless tube; to allow forming an underwear or undergarment of various types or shapes or structures, such as, briefs, Tanga, String, Hip-Hugger, Boxer Short, or other undergarments having a back-gusset seam.

In accordance with some embodiments, only the Passepartout member is non-removably attached to the underwear, keeping the Pad at exactly the place where it should be, and maintaining the seal of the frames around the leak-proof materials; yet allowing to use techniques of sewing, bonding, and/or ultrasonic treatment; and also allowing to avoid a front-side seam of the underwear (as it may be kept open in the front).

Some embodiments may include a Seamless product, or may be produced in a seamless technology or production technique; to produce a free-flow Pad in a manner that allows the pad to be integrated be integrated into various forms of Seamless underwear; that are knitted in (or around, or by using) a tube or a generally cylindrical template, without any side-seams; or using a body size-cylinder on a suitable knitting machine (e.g., a Santoni machine).

Some embodiments may optionally include, or may be used in conjunction with, a variety of garments or undergarments; for example, female underwear, men underwear, panties, lingerie items, swimsuit items, sleepwear items, sportswear items, pants, leggings, yoga pants, elastic pants, non-elastic pants or tailored pants, Bikini underwear, Tanga underwear, String Tanga underwear, Shorts underwear, Boxer Shorts underwear, slip underwear, sport skirts, sport pants, seamless bottom garments, seamless bottom undergarments, or the like.

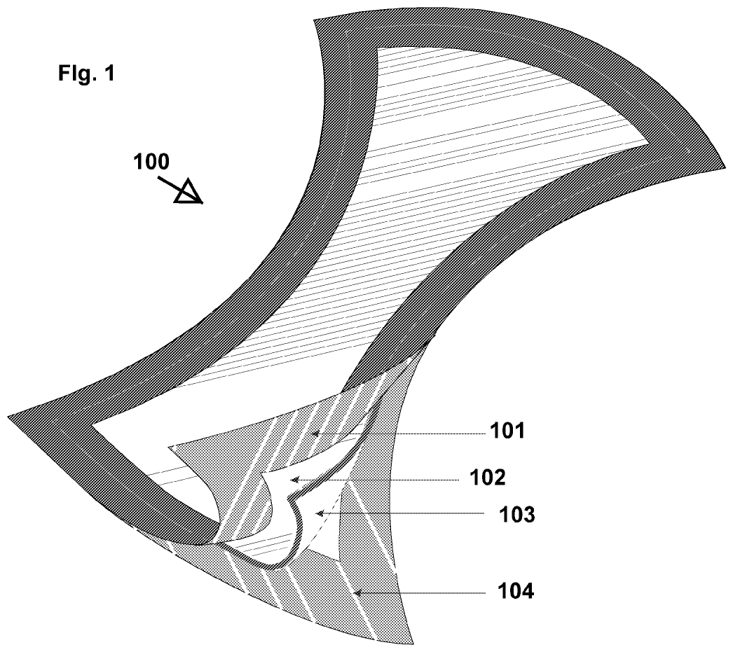

Reference is made to FIG. 1, which is a schematic illustration of a multiple-layer fabric-made pad 100, in accordance with some embodiments.

Element 101=Top Layer: Passepartout frame film-adhesive membrane.

Element 102=Middle Layers: (upper side) high wicking dry touch fabric.

Element 103=Middle Layers: (bottom side) high absorbance and water-proof membrane to prevent leakage.

Element 104=Bottom Layer: Passepartout frame film-adhesive membrane.

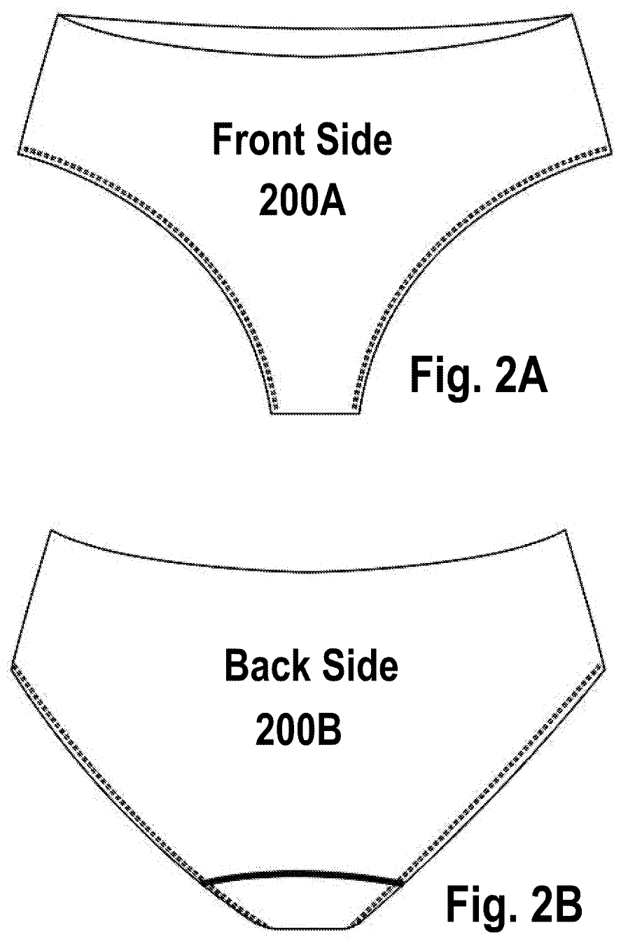

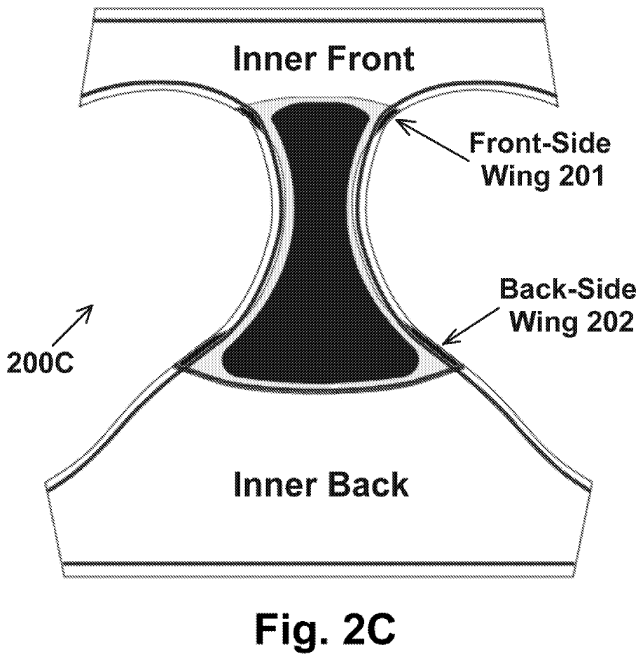

Reference is made to FIGS. 2A-2C, which are schematic illustrations of several views of an underwear (e.g., intimate briefs) for females, in accordance with some embodiments. FIGS. 2A-2C show, for demonstrative purposes, a small side-seam, which is optional and which need not appear and need not be in a Seamless garment. FIG. 2A shows a front view 200A; FIG. 2B shows a back view (or a rear view) 200B; and FIG. 2C shows an inner view 200C or an inner-side view of the central region (or crotch-covering region, or gusset region).

Front side: No visible front seam to hold the Passepartout technology Pad.

Back side: Visible sewing line at the end of a crotch, attaching the open Seamless-Tube and at the same time stitching into the Passepartout extension to hold the Pad in place.

Inner side: Skin facing High-Wicking Fabric, without any Anti-Bacterial Finishing; extension of the Passepartout allowing partial and/or full stitching around (or near) the leg opening, and attaching the back-gusset seam.

In some embodiments, the inner-front region has no seam, to allow ‘invisible attachment’ on the front of the underwear.

FIG. 2C shows an inner-side view of the Passepartout element (or Passepartout margin or Passepartout frame or Passepartout holding element or Passepartout bordering element), which holds therein the functional leak-proof fabric-made (or fabric-based) pad (e.g., by holding such pad, such as, by holding in place the borders or edges of such pad beneath a top-layer of the Passepartout element). The front region or the front-facing region of the inner side may allow or may provide increased breathability, and may avoid stitching or seams or hems in order to avoid a visible gusset at the front side, and/or to provide wearer comfort and a clean front-side look.

In some embodiments, optionally, Passepartout wings or wing members (201 and 202) or additional strips of fabric or fabric extensions may allow full or partial sewing of the Passepartout element to (or near, or at) the leg openings, to provide additional connection support to the Passepartout relative to the leg opening regions of the underwear.

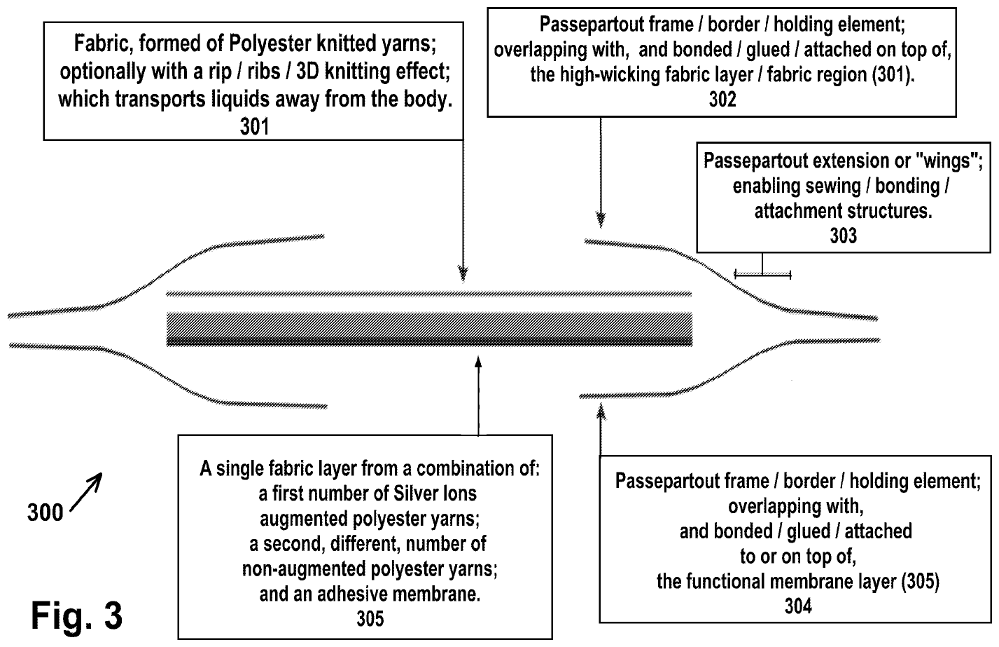

Reference is made to FIG. 3, which is a schematic illustration of a fabric-based leak-proof pad 300, demonstrating the Passepartout element or member, which enables to “sandwich” or trap therein, or hold in place securely, two (or more) independent fabric layers, and leaving an area for stitching into (or for otherwise attaching or connecting to) the seamless undergarment or garment.

Element 301=Middle Layers, including: (upper side) high wicking dry touch fabric, and (bottom side) high absorbance and water-proof membrane to prevent leakage.

Element 302=Top Layer: Passepartout frame film-adhesive membrane.

Element 303=Area/region/border/margin, for stitching/ultrasonic treatment/bonding of the pad to the undergarment or garment.

Element 304=Bottom Layer: Passepartout frame film-adhesive membrane.

Element 305=Fabric element or fabric-based element or fabric-based layer; knitted from a combination of, for example, (i) a first number of Polyester yarn(s), and (ii) a second number of silver-augmented yarn(s) or ionized silver yarn(s) or silver-containing yarn(s) or silver-ions containing yarn(s), and an Adhesive Membrane, all made or integrated or laminated into as single fabric-made layer.

In some embodiments, the entirety of the underwear may be seamless; or, at least 90 or 80 or 75 or 66 or 50 percent the fabric-area of the underwear that directly touches the human wearer is seamless.

In some embodiments, the entirety of the fabric-made leak-protection pad may be seamless; or, at least 90 or 80 or 75 or 66 or 50 percent the fabric-area of the fabric-made leak-protection pad that directly touches the human wearer is seamless.

The present invention further comprises a method or process, which may be computer-assisted or machine-assisted, or may be implemented via machines and/or manufacturing equipment (e.g., sewing machine, cutting machine, machine-controlled needles or cutters, robotic arms, conveyor belts, or the like). The method may comprise, for example: producing a three-dimensional ribbed accordion structure of a pouch; and connecting it to, or incorporating it as integral part of, an underwear for men.

For demonstrative purposes, some portions of the discussion herein may relate to an underwear; however, the present invention may similarly comprise other types of garments and/or undergarments that may have similar properties as the described underwear; for example, swimwear, swimsuit, shorts, athletic wear, sports-wear, boxers, briefs, knitted boxers, knitted briefs, thongs, cycling shorts, fitness shorts, shorts used by athletes, lingerie items, sleepwear, swimwear, or the like.

Some embodiments provide an underwear for a female wearer, the underwear comprising: a rear-side fabric-region that is configured to cover at least some of buttocks of the female wearer; a front-side fabric-region that is generally opposite said rear-side fabric-region and that is configured to cover at least some of a pelvis of the female wearer; and a gusset (or crotch-covering, or genitalia-covering) fabric-region that is configured to cover genitalia and/or crotch of the female wearer. The body-facing side of the gusset fabric-region comprises a fabric-based Passepartout frame element which holds in place therein a fabric-based leak-protection pad that absorbs fluids and prevents fluid leaks.

In some embodiments, the fabric-based Passepartout frame element comprises a fabric-based border element, having an inner aperture that has an area that is smaller than an area of the fabric-based leak-protection pad, to enable said fabric-based border element of said fabric-based Passepartout frame element to hold in place the fabric-based leak-protection pad.

In some embodiments, the fabric-based leak-protection pad comprises at least a fabric-portion that is a Silver Ionized fabric-region that was treated with or augmented with Silver Ions.

In some embodiments, the fabric-based leak-protection pad comprises at least a fabric-portion that is a fabric-region that was treated with an anti-bacterial agent.

In some embodiments, the fabric-based leak-protection pad comprises at least a fabric-portion that is a fabric-region that was treated with an odors-absorbing agent.

In some embodiments, the fabric-based Passepartout frame element holds in place the fabric-based leak-protection pad in a user-removable manner; wherein the fabric-based leak-protection pad is manually removable from within the fabric-based border element of the fabric-based Passepartout frame element.

In some embodiments, the fabric-based leak-protection pad is a multiple-use fabric-based leak-protection pad that can be washed or laundered and then dried, and is then insertable back into the fabric-based border element of the fabric-based Passepartout frame element.

In some embodiments, the fabric-based leak-protection pad is a single-use fabric-based leak-protection pad that is removable and disposable, and can be manually removed from the fabric-based Passepartout frame element and discarded to enable insertion of another fabric-based leak-protection pad into the fabric-based border element of the fabric-based Passepartout frame element.

In some embodiments, the fabric-based Passepartout frame element integrally and securely holds in place the fabric-based leak-protection pad in a non-removable manner; wherein the fabric-based leak-protection pad is non-removable from within the fabric-based border element of the fabric-based Passepartout frame element; wherein the fabric-based leak-protection pad continuously remains integrally attached to the fabric-based border element of the fabric-based Passepartout frame element.

In some embodiments, the fabric-based leak-protection pad comprises at least one leakage-protection membrane for absorbing fluids of the female wearer.

In some embodiments, the fabric-based leak-protection pad comprises: a top-side, body-facing, body-touching, leakage-protection membrane, configured to absorb for absorbing fluids of the female wearer; a bottom-side, non body-touching, leakage-protection membrane, facing away from the female wearer, and not directly touching the female wearer; a sandwiched fabric-based layer, that is sandwiched or trapped between the top-side leakage-protection membrane and the bottom-side leakage-protection membrane, which is not directly touching the female wearer.

In some embodiments, the sandwiched fabric-based layer, and only the sandwiched fabric-based layer, comprises molecules of Ionized Silver.

In some embodiments, the sandwiched fabric-based layer, and only the sandwiched fabric-based layer, comprises an anti-bacterial agent.

In some embodiments, the sandwiched fabric-based layer, and only the sandwiched fabric-based layer, comprises an odor-absorbing agent.

In some embodiments, the bottom-side, non body-touching, leakage-protection membrane is, or comprises: a foil that is integrally attached and is non-removably laminated to a bottom-side of said sandwiched fabric-based layer.

In some embodiments, the bottom-side, non body-touching, leakage-protection membrane is, or comprises: a foil that is integrally attached and is non-removably laminated to a top-side of said sandwiched fabric-based layer.

In some embodiments, the fabric-based Passepartout frame element is entirely seam-less, and is connected to said gusset fabric-region via a seam-less connection.

In some embodiments, at least an entirety of a body-facing side of the fabric-based Passepartout frame element, is seam-less and does not have any seams and/or stitches that touch a body of the female wearer.

In some embodiments, the fabric-based leak-protection pad is entirely seam-less, and does not have any seams that touch a body of the female wearer.

In some embodiments, at least an entirety of a body-facing side of the fabric-based leak-protection pad, is seam-less and does not have any seams and/or stitches that touch a body of the female wearer.

In some embodiments, the fabric-based leak-protection pad contains therein, trapped yarns that are trapped within the fabric-based leak-protection pad and that do not touch a skin of the female wearer, wherein the trapped yarns comprise yarns that are pre-treated or pre-augmented with Ionized Silver molecules.

In some embodiments, the fabric-based Passepartout frame element, is integrally and non-removably connected to an inner side of said underwear via a seam-less, stitch-less, lamination-based connection.

In some embodiments, the fabric-based Passepartout frame element, is integrally and non-removably connected to an inner side of said underwear via a seam-less, stitch-less, glue-based connection.

In some embodiments, a top-side of the fabric-based Passepartout frame element is formed of a high-wicking fabric; wherein a bottom-side of the fabric-based Passepartout frame element comprises an adhesive foil that is integrally laminated to an inner-side of said underwear.

In some embodiments, the fabric-based leak-protection pad comprises at least: a top-side, fast-wicking, knitted fabric layer, that has integrally-knitted fabric rips or fabric ribs or three-dimensional fabric craters or three-dimensional fabric protrusions, and that rapidly collects fluids of the female wearer and rapidly transports at least some of said fluids downwardly away from the female wearer towards a fluid-absorbing membrane that is connected beneath said top-side fast-wicking knitted fabric layer of said fabric-based leak-protection pad.

In some embodiments, the fabric-based leak-protection pad comprises: augmented yarns, which are polyester yarns that are augmented pre-treated or pre-augmented with Ionized Silver molecules; non-augmented yarns, which are polyester yarns are not augmented and are not pre-treated with Ionized Silver molecules or with other anti-bacterial agent. In some embodiments, only the non-augmented yarns, and not the augmented yarns, are in touch with a skin of the female wearer; wherein the augmented yarns are trapped or sandwiched or encapsulated within non-augmented yarns of said fabric-based leak-protection pad.

In some embodiments, a ratio of (i) a number of the augmented yarns, to (ii) a number of the non-augmented yarns, is in a range of 0.50 to 2.0.

In some embodiments, a number of the augmented yarns in said fabric-based leak-protection pad, is greater than a number of the non-augmented yarns in said fabric-based leak-protection pad.

In some embodiments, a number of the augmented yarns in said fabric-based leak-protection pad, is smaller than a number of the non-augmented yarns in said fabric-based leak-protection pad.

In some embodiments, said fabric-based leak-protection pad is fleece-less and lacks any fleece element and/or any fleece region.

In some embodiments, the fabric-made leak-protection pad comprises at least a Silver Ionized fabric structure that is knitted from (i) one or more polyester yarns, and (ii) one or more yarns that are augmented or treated with Ionized Silver molecules; wherein said Silver Ionized fabric structure does not directly touch a skin of the female wearer; wherein said Silver Ionized fabric structure is sandwiched between (i) a top-side fabric layer that is in direct touch with a skin of the female wearer, and (ii) a bottom-side leakage-protection membrane or foil that is non-removably laminated to said Silver Ionized fabric structure.

In some embodiments, the underwear and/or the pad and/or the fabric-based Passepartout frame element are not formed of towel knitting, and are not towel-liked knitted, and exclude any tower-like knits or Terry knits; and/or include only non-Terry cloth and/or non-Terry knitted elements.

Some embodiments provide a leak-proof seamless underwear having a fabric-made leak-protection pad. For example, an underwear for a female wearer includes: a rear-side fabric-region that is configured to cover at least some of buttocks of the female wearer; a front-side fabric-region that is generally opposite said rear-side fabric-region and that is configured to cover at least some of a pelvis of the female wearer; and a gusset (or crotch-covering) fabric-region that is configured to cover genitalia and/or crotch of the female wearer. A body-facing or inner side of the gusset fabric-region includes a fabric-based Passepartout frame element which holds in place therein a fabric-based leak-protection pad that absorbs fluids and prevents fluid leaks.

Functions, operations, components and/or features described herein with reference to one or more embodiments, may be combined with, or may be utilized in combination with, one or more other functions, operations, components and/or features described herein with reference to one or more other embodiments, or vice versa.

While certain features of some embodiments have been illustrated and described herein, many modifications, substitutions, changes, and equivalents may occur to those skilled in the art. Accordingly, the claims are intended to cover all such modifications, substitutions, changes, and equivalents.

1. An underwear for a female wearer, the underwear comprising:
a rear-side fabric-region that is configured to cover at least some of buttocks of the female wearer;
a front-side fabric-region that is generally opposite said rear-side fabric-region and that is configured to cover at least some of a pelvis of the female wearer;
a gusset fabric-region that is configured to cover genitalia and/or crotch of the female wearer;
wherein a body-facing side of the gusset fabric-region, comprises a fabric-based Passepartout frame element which holds in place therein a fabric-based leak-protection pad that absorbs fluids and prevents fluid leaks.

2. The underwear of claim 1,
wherein the fabric-based Passepartout frame element comprises a fabric-based border element, having an inner aperture that has an area that is smaller than an area of the fabric-based leak-protection pad, to enable said fabric-based border element of said fabric-based Passepartout frame element to hold in place the fabric-based leak-protection pad.

3. The underwear of claim 2,
wherein the fabric-based leak-protection pad comprises at least a fabric-portion that is a Silver Ionized fabric-region that was treated with or augmented with Silver Ions.

4. The underwear of claim 2,
wherein the fabric-based leak-protection pad comprises at least a fabric-portion that is a fabric-region that was treated with an anti-bacterial agent.

5. The underwear of claim 2,
wherein the fabric-based leak-protection pad comprises at least a fabric-portion that is a fabric-region that was treated with an odors-absorbing agent.

6. The underwear of claim 2,
wherein the fabric-based Passepartout frame element holds in place the fabric-based leak-protection pad in a user-removable manner,
wherein the fabric-based leak-protection pad is manually removable from within the fabric-based border element of the fabric-based Passepartout frame element.

7. The underwear of claim 6,
wherein the fabric-based leak-protection pad is a multiple-use fabric-based leak-protection pad that can be washed or laundered and then dried, and is then insertable back into the fabric-based border element of the fabric-based Passepartout frame element.

8. The underwear of claim 6,
wherein the fabric-based leak-protection pad is a single-use fabric-based leak-protection pad that is removable and disposable, and can be manually removed from the fabric-based Passepartout frame element and discarded to enable insertion of another fabric-based leak-protection pad into the fabric-based border element of the fabric-based Passepartout frame element.

9. The underwear of claim 2,
wherein the fabric-based Passepartout frame element integrally and securely holds in place the fabric-based leak-protection pad in a non-removable manner,
wherein the fabric-based leak-protection pad is non-removable from within the fabric-based border element of the fabric-based Passepartout frame element,
wherein the fabric-based leak-protection pad continuously remains integrally attached to the fabric-based border element of the fabric-based Passepartout frame element.

10. The underwear of claim 2,
wherein the fabric-based leak-protection pad comprises at least one leakage-protection membrane for absorbing fluids of the female wearer.

11. The underwear of claim 2,
wherein the fabric-based leak-protection pad comprises:
a top-side, body-facing, body-touching, leakage-protection membrane, configured to absorb for absorbing fluids of the female wearer;
a bottom-side, non body-touching, leakage-protection membrane, facing away from the female wearer, and not directly touching the female wearer;
a sandwiched fabric-based layer, that is sandwiched or trapped between the top-side leakage-protection membrane and the bottom-side leakage-protection membrane, which is not directly touching the female wearer.

12. The underwear of claim 11,
wherein the sandwiched fabric-based layer, and only the sandwiched fabric-based layer, comprises molecules of Ionized Silver.

13. The underwear of claim 11,
wherein the sandwiched fabric-based layer, and only the sandwiched fabric-based layer, comprises an anti-bacterial agent.

14. The underwear of claim 11,
wherein the sandwiched fabric-based layer, and only the sandwiched fabric-based layer, comprises an odor-absorbing agent.

15. The underwear of claim 11,
wherein the bottom-side, non body-touching, leakage-protection membrane is, or comprises: a foil that is integrally attached and is non-removably laminated to a bottom-side of said sandwiched fabric-based layer.

16. The underwear of claim 11,
wherein the bottom-side, non body-touching, leakage-protection membrane is, or comprises: a foil that is integrally attached and is non-removably laminated to a top-side of said sandwiched fabric-based layer.

17. The underwear of claim 2,
wherein the fabric-based Passepartout frame element is entirely seam-less, and is connected to said gusset fabric-region via a seam-less connection.

18. The underwear of claim 2,
wherein at least an entirety of a body-facing side of the fabric-based Passepartout frame element, is seam-less and does not have any seams and/or stitches that touch a body of the female wearer.

19. The underwear of claim 18,
wherein the fabric-based leak-protection pad is entirely seam-less, and does not have any seams that touch a body of the female wearer.

20. The underwear of claim 18,
wherein at least an entirety of a body-facing side of the fabric-based leak-protection pad, is seam-less and does not have any seams and/or stitches that touch a body of the female wearer.

21. The underwear of claim 2,
wherein the fabric-based leak-protection pad contains therein: trapped yarns that are trapped within the fabric-based leak-protection pad and that do not touch a skin of the female wearer, wherein the trapped yarns comprise yarns that are pre-treated or pre-augmented with Ionized Silver molecules.

22. The underwear of claim 2,
wherein the fabric-based Passepartout frame element, is integrally and non-removably connected to an inner side of said underwear via a seam-less, stitch-less, lamination-based connection.

23. The underwear of claim 2,
wherein the fabric-based Passepartout frame element is integrally and non-removably connected to an inner side of said underwear via a seam-less, stitch-less, glue-based connection.

24. The underwear of claim 2,
wherein a top-side of the fabric-based Passepartout frame element is formed of a high-wicking fabric;
wherein a bottom-side of the fabric-based Passepartout frame element comprises an adhesive foil that is integrally laminated to an inner-side of said underwear.

25. The underwear of claim 2,
wherein the fabric-based leak-protection pad comprises at least:
a top-side, fast-wicking, knitted fabric layer,
that has integrally-knitted fabric rips or fabric ribs or three-dimensional fabric craters or three-dimensional fabric protrusions,
and that rapidly collects fluids of the female wearer and rapidly transports at least some of said fluids downwardly away from the female wearer towards a fluid-absorbing membrane that is connected beneath said top-side fast-wicking knitted fabric layer of said fabric-based leak-protection pad.

26. The underwear of claim 2,
wherein the fabric-based leak-protection pad comprises:
augmented yarns, which are polyester yarns that are augmented pre-treated or pre-augmented with Ionized Silver molecules;
non-augmented yarns, which are polyester yarns are not augmented and are not pre-treated with Ionized Silver molecules or with other anti-bacterial agent.

27. The underwear of claim 26,
wherein only the non-augmented yarns, and not the augmented yarns, are in touch with a skin of the female wearer;
wherein the augmented yarns are trapped or sandwiched or encapsulated within non-augmented yarns of said fabric-based leak-protection pad.

28. The underwear of claim 27,
wherein a ratio of
(i) a number of the augmented yarns,
to
(ii) a number of the non-augmented yarns,
is in a range of 0.50 to 2.0.

29. The underwear of claim 27,
wherein a number of the augmented yarns in said fabric-based leak-protection pad is greater than
a number of the non-augmented yarns in said fabric-based leak-protection pad.

30. The underwear of claim 27,
wherein a number of the augmented yarns in said fabric-based leak-protection pad is smaller than
a number of the non-augmented yarns in said fabric-based leak-protection pad.

31. The underwear of claim 2,
wherein said fabric-based leak-protection pad is fleece-less and lacks any fleece element or any fleece region.

32. The underwear of claim 2,
wherein the fabric-made leak-protection pad comprises at least a Silver Ionized fabric structure that is knitted from (i) one or more polyester yarns, and (ii) one or more yarns that are augmented or treated with Ionized Silver molecules;
wherein said Silver Ionized fabric structure does not directly touch a skin of the female wearer,
wherein said Silver Ionized fabric structure is sandwiched between (i) a top-side fabric layer that is in direct touch with a skin of the female wearer, and (ii) a bottom-side leakage-protection membrane or foil that is non-removably laminated to said Silver Ionized fabric structure.
